(19) United States
(12) Patent Application Publication
(10) Pub. No.: US 2014/0194615 A1
(43) Pub. Date: Jul. 10, 2014

(54) METHOD FOR THE PREPARATION OF 2-[4-[(METHYLAMINO)CARBONYL]-1-H-PYRAZOL-1-YL]ADENOSINE MONOHYDRATE

(71) Applicant: FARMAK, A.S., Olomouc (CZ)

(72) Inventors: Lubomir Kvapil, Slatinice (CZ); Pavel Hradil, Hlusovice (CZ); Martin Grepl, Hlusovice (CZ); Petr Slezar, Olomouc (CZ); Barbora Dvorakova, Most 1 (CZ)

(21) Appl. No.: 14/239,788

(22) PCT Filed: Aug. 14, 2012
(86) PCT No.: PCT/CZ2012/000080

§ 371 Date: Feb. 20, 2014

(30) Foreign Application Priority Data
Aug. 22, 2011 (CZ) PV 2011-517

(51) Int. Cl. C07H 19/16

(52) U.S. Cl. CPC C07H 19/16
USPC 536/27.61

(57) ABSTRACT

A method for the preparation of 2-[4-[(methylamino)carbonyl]-1-H-pyrazol-1-yl]adenosine monohydrate of formula I by reaction of 2-(4-methoxycarbonylpyrazol-1-yl) adenosine of formula III with a solution of methylamine in a non-aqueous solvent, optionally in combination with another inert solvent, to produce anhydrous 2-[4-[(methylamino)carbonyl]-1-H-pyrazol-1-yl]adenosine, which is converted to 2-[4-[(methylamino)carbonyl]-1-H-pyrazol-1-yljadenosine monohydrate of formula I by addition of water.

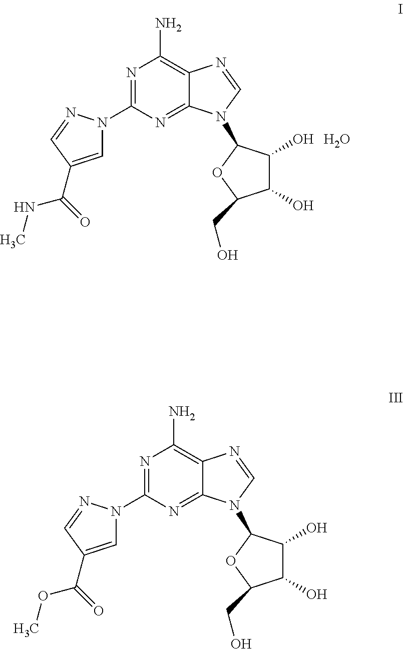

### TECHNICAL FIELD

The invention relates to a new preparation method of 2-[4-[(methylamino)carbonyl]-1-H-pyrazol-1-yl]adenosine monohydrate of formula I,

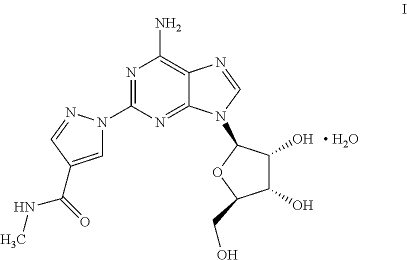 [SMILES: CNC(=O)c1cnn(-c2nc(N)c3ncn([C@@H]4O[C@H](CO)[C@@H](O)[C@H]4O)c3n2)c1 ; "· H2O"]

which is known as Regadenoson and is used as a coronary vasodilator for diagnostic purposes during radionuclide examinations of the heart.

### BACKGROUND ART

The methods of preparation of I that are known so far are based on a reaction of 2-(4-ethoxycarbonylpyrazol-1-yl)adenosine of formula II

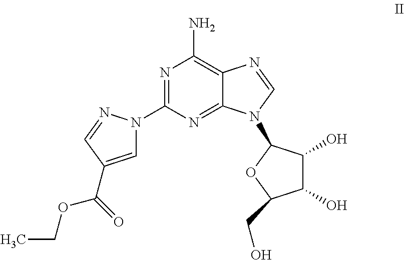 [SMILES: CCOC(=O)c1cnn(-c2nc(N)c3ncn([C@@H]4O[C@H](CO)[C@@H](O)[C@H]4O)c3n2)c1]

with an aqueous solution of methylamine.

Literature mentions a reaction of 2-(4-ethoxycarbonylpyrazol-1-yl)adenosine of formula II with a 40% solution of methylamine in water at 65° C. for 24 hours with the yield of 75% (J. Zablocki et al.: Nucleosides, Nucleotides and Nucleic Acids 2001, 20(4-7) 343, or U.S. Pat. No. 6,403,567).

Another well-known embodiment uses a reaction of 2-(4-ethoxycarbonylpyrazol-1-yl)adenosine of formula II with a 40% solution of methylamine in water at the laboratory temperature for 4 hours, subsequent removal of the excess of methylamine at a reduced pressure, cooling of the reaction mixture and removal of the product in the yield of 78.4% and purity of 99.6% (HPLC) (WO 2007/092372 and U.S. Pat. Appln. 2010/0267953).

A very similar process of a reaction of 2-(4-ethoxycarbonylpyrazol-1-yl)adenosine of formula II with a 40% solution of methylamine in water at the laboratory temperature for 4 hours, concentration by removal of the excess of methylamine, cooling to 0° C. for 2 hours and subsequent isolation is described in the patent application WO 2008/143667 without mentioning the yield or purity.

Literature also mentions the possibility of synthesis of derivatives of I by means of a cross-coupling reaction between 2-iodoadenosine and derivatives of 4-pyrazole carboxylic acid (Drugs of the Future 2004, 29 (10), 998, and in the patent U.S. Pat. No. 6,514,949). However, this synthesis is not sufficiently documented with experimental data, but what can be assumed is that complexes with heavy metals are used in this case and the synthesized derivative has then to be laboriously (chromatographically) purified.

Disadvantages of the above mentioned methods:
Both the starting compound 2-(4-ethoxycarbonylpyrazol-1-yl)adenosine of formula II and the product 2-[4-[(methylamino)carbonyl]-1-H-pyrazol-1-yl]adenosine of formula I are poorly soluble in water, an aqueous solution of methylamine and in common organic solvents.
Therefore, the reaction according to the above mentioned methods proceeds in a suspension that contains a mixture of both the starting 2-(4-ethoxycarbonylpyrazol-1-yl)adenosine of formula II and the product 2-[4-[(methylamino)carbonyl]-1-H-pyrazol-1-yl]adenosine of formula I; experts in the art are aware that in such cases incomplete conversion of the starting compound to the product may occur.
Another disadvantage consists in the use of an aqueous solution of methylamine since it has been found that water may also get involved in the reaction of the ester and the aqueous solution of the amine, which may produce, as an impurity, the corresponding acid, or a salt thereof with the amine used (see J. March: Advanced Organic Chemistry, J. Wiley Interscience Publ., 4th Edition 1992, page 424).

### DISCLOSURE OF INVENTION

The above mentioned disadvantages, especially the disadvantages related to carrying the reaction out in a suspension and to use of aqueous methylamine have been eliminated by the method according to the invention, which is a method for the preparation of 2-[4-[(methylamino)carbonyl]-1-H-pyrazol-1-yl]adenosine of formula I characterized by a reaction of 2-(4-methoxycarbonylpyrazol-1-yl)adenosine of formula III

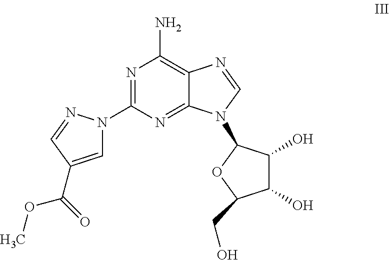 [SMILES: COC(=O)c1cnn(-c2nc(N)c3ncn([C@@H]4O[C@H](CO)[C@@H](O)[C@H]4O)c3n2)c1]

with methylamine in a non-aqueous solvent.

An organic solvent from the group of alcohols such as methanol and ethanol, preferably methanol, or a solvent from the group of polar aprotic solvents, preferably dimethyl sulfoxide, can be used as the non-aqueous solvent of methylamine.

Non-aqueous solutions of methylamine have been surprisingly found to dissolve both the starting 2-(4-methoxycarbonylpyrazol-1-yl)adenosine of formula III and the produced 2-[4-[(methylamino)carbonyl]-1-H-pyrazol-1-yl]adenosine much more easily than a solution of methylamine in water. Another aspect of the invention is carrying the reaction out in a combination with another inert solvent, which is used to dissolve 2-(4-methoxycarbonylpyrazol-1-yl)adenosine of formula III prior to its reaction with methylamine in the non-aqueous solvent. Such other inert solvents can include, in particular, solvents from the group of polar aprotic solvents, preferably dimethyl sulfoxide.

After the reaction is complete, the excessive methylamine is removed in vacuo, which reduces solubility of the resulting 2-[4-[(methylamino)carbonyl]-1-H-pyrazol-1-yl]adenosine, which can precipitate in a gel-like anhydrous form in some cases. The latter converts, by addition of water, to the resulting 2-[4-[(methylamino)carbonyl]-1-H-pyrazol-1-yl]adenosine monohydrate of formula I.

As the aminolysis proceeds in an anhydrous environment, the main competing reaction—hydrolysis and formation of an acid—does not occur. However, after the reaction is complete, water has to be added to convert the very poorly isolable anhydrous form of regadenoson to the well filterable monohydrate of formula I. On the other hand, there is no longer any risk of the secondary reaction in this phase.

The reaction in accordance with the present invention can be carried out in a wide range of temperatures, preferably especially at the laboratory temperature, but also at slightly elevated temperatures of up to ca. 50° C. in closed containers.

As some of the above mentioned methods (WO 2007/092372) do not mention the achieved yields or purity, a reproduction of the method of the above mentioned application was performed. The results are summarized in the overview below and the results of Example 1 of the present invention have been added for comparison.

TABLE 1

| | | | HPLC | | |
|---|---|---|---|---|---|
| | | Yield | purity | Impurities | |
| | | [%] | [%] | “ester” [%] | “acid” [%] |
| 1 | PCTWO2007/092372, | 71.4 | 96.8 | 2.74 | 0.33 |
| | Example 4 (reaction | | | | |
| | time: 4 hours) | | | | |
| 2 | PCTWO2007/092372, | 74.3 | 98.2 | 1.40 | 0.24 |
| | Example 4 (reaction | | | | |
| | time: 24 hours) | | | | |
| 3 | Present invention, | 86.5 | 99.9 | 0.03 | 0.03 |
| | Example 1 | | | | |

The starting 2-(4-ethoxycarbonylpyrazol-1-yl)adenosine of formula II and 2-(4-methoxycarbonylpyrazol-1-yl)adenosine of formula III were used in the same quality (HPLC purity 99.8%) for all the experiments.

It can be seen from the table that during the reproduction of the method of the application WO 2007/092372; Example 4, wherein the reaction time of 4 hours mentioned in the application was exactly kept, the yield of 71.4% and HPLC purity of 96.8% was achieved, while 2.74% of the “ester” (i.e., 2-(4-ethoxycarbonylpyrazol-1-yl)adenosine of formula II) remained unreacted, and 0.33% of the “acid”, or its methylamine salt, appeared as an impurity produced by reaction with water, which is surprisingly very difficult to remove. Therefore, the reaction time was extended to 24 hours, which slightly increased both the yield (74.3%) and the purity (98.2%, HPLC), but there still remained the quite high amount of 1.40% of unreacted 2-(4-ethoxycarbonylpyrazol-1-yl)adenosine of formula II.

On the other hand, the method according to the present invention provided a purity of 99.9% HPLC and only 0.03% of 2-(4-methoxycarbonylpyrazol-1-yl)adenosine of formula III remained unreacted, and also the amount of the poorly removable “acid” impurity is remarkably lower (0.03%).

In the manufacture of drug substances and diagnostic products it is the quality or purity of the final product, as well as purity of intermediates, that is the most important parameter (see, e.g., ICH Harmonized Tripartite Guideline, Impurities in New Drug Substances Q3A(R2), 2006). The purity achieved according to WO 2007/092372 amounted to about 98% (HPLC), but only the method according to the present invention provided a purity over 99% (HPLC). This means that only a product according to the present invention does not require re-purifying, e.g., by crystallization, while the products prepared according to WO 2007/092372 will probably additionally require some re-purification.

The advantages of the method according to the present invention are as follows:
The reaction proceeds in a homogeneous solution, which ensures easy control of the course of the reaction and its high conversion with minimum amounts of impurities
The reaction proceeds under moderate reaction conditions
Higher purity and yield result.

### EXAMPLES

The essence of carrying out the invention is clarified in a more detailed way in the following examples. These examples only have an illustrative character and do no limit the scope of the invention in any way.

Differential Scanning Calorimetry (DSC) was measured using Perkin Elmer instrumentation, the Pyris Diamond DSC model with evaluation using the Pyris software, version 5.0.

The samples were analyzed in open aluminium pans in a nitrogen atmosphere.

### Example 1

A suspension of 1 g of 2-(4-methoxycarbonylpyrazol-1-yl)adenosine (2.556 mmol) in 10 ml of 40% methylamine in methanol is stirred in a closed flask at 20° C. until a solution is obtained (for ca. 3 to 5 hours). The resulting solution is left to stand at the above mentioned temperature for another 15 hours. The solution is then filtered with active carbon and the filtrate is carefully slightly concentrated, while a gel-like precipitate of anhydrous 2-[4-[(methylamino)carbonyl]-1-H-pyrazol-1-yl]adenosine results. Slow addition of 10 ml of water converts the gel-like precipitate to fine powdery precipitate, which is, after stirring up, filtered with suction, thoroughly washed with water, then with methanol and dried in vacuo until a constant weight.

This procedure provides 0.9 g of 2-[4-[(methylamino)carbonyl]-1H-pyrazol-1-yl]adenosine monohydrate, i.e. 86.5%, with the purity of 99.9% (HPLC).

The Differential Scanning Calorimetry (DSC) exhibits endo transitions at 177° C. and 188° C.

### Example 2

A suspension of 2 g of 2-(4-methoxycarbonylpyrazol-1-yl)adenosine (5.111 mmol) and 6 ml of DMSO is heated up to ca. 50 ° C. while being stirred. 14 ml of 40% methylamine in methanol are added to the resulting solution after cooling to 20° C. The reaction solution is then left to stand in a closed flask at 20° C. for 20 hours. The solution is then filtered with active carbon and the filtrate is carefully slightly concentrated, while a gel-like precipitate of anhydrous 2-[4-[(methylamino)carbonyl]-1-H-pyrazol-1-yl]adenosine results. Slow addition of 20 ml of water converts the gel-like precipitate to fine powdery precipitate, which is, after stirring up, filtered with suction, thoroughly washed with water, then with methanol and dried in vacuo until a constant weight.

This procedure provides 1.69 g of 2-[4-[(methylamino)carbonyl]-1H-pyrazol-1-yl]adenosine monohydrate, i.e., 80.9%, with the HPLC purity of 99.6%.

### Example 3

A suspension of 1 g of 2-(4-methoxycarbonylpyrazol-1-yl)adenosine (2.556 mmol) in 10 ml of 40% methylamine in ethanol is stirred in a pressure tube in a bath of 50° C. During ca. 4 hours a solution results, which is stirred at the above mentioned temperature for another 8 hours. Then the reaction solution is cooled, filtered with active carbon and the filtrate is slightly concentrated in vacuo, while a gel-like precipitate of anhydrous 2-[4-[(methylamino)carbonyl]-1-H-pyrazol-1-yl]adenosine results. Slow addition of 8 ml of water produces fine powdery precipitate, which is, after stirring up, filtered with suction, thoroughly washed with water, then with methanol and dried in vacuo until a constant weight.

This procedure provides 0.9 g of 2-[4-[(methylamino)carbonyl]-1H-pyrazol-1-yl]adenosine monohydrate, i.e. 86.3%, with the purity of 99.6% (HPLC).

### Example 4

1.3 g of 2-(4-methoxycarbonylpyrazol-1-yl)adenosine (3.322 mmol) are added to 8 ml of 20% methylamine in dimethyl sulfoxide. The resulting solution is stirred in a pressure tube in a bath of 50° C. for 12 hours. Then the reaction solution is cooled, filtered with active carbon and the filtrate is slightly concentrated in vacuo. 15 ml of water are then carefully added to the reaction solution. The resulting fine powdery precipitate is then filtered with suction, washed with water, then with methanol and dried in vacuo until a constant weight.

This procedure provides 1.15 g of 2-[4-[(methylamino)carbonyl]-1H-pyrazol-1-yl]adenosine monohydrate, i.e., 84.6%, with the purity of 99.4% HPLC.

### INDUSTRIAL APPLICABILITY

The method for the preparation of 2-[4-[(methylamino)carbonyl]-1H-pyrazol-1-yl]adenosine monohydrate of formula I according to the present invention can be applied in convenient technological and economic conditions, maintaining at the same time high purity of the product and higher yield and purity, all this under moderate reaction conditions.

1. A method for the preparation of 2-[4-[(methylamino)carbonyl]-1-H-pyrazol-1-yl]adenosine monohydrate of formula I 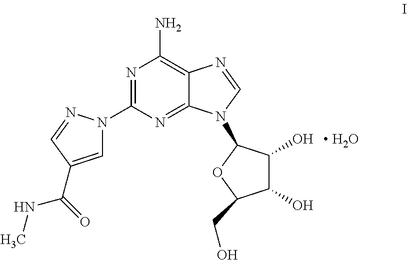 [SMILES: CNC(=O)c1cnn(-c2nc(N)c3ncn([C@@H]4O[C@H](CO)[C@@H](O)[C@H]4O)c3n2)c1 ; "· H2O"]
comprising the steps of:
a) reacting 2-(4-methoxycarbonylpyrazol-1-yl)adenosine of formula III 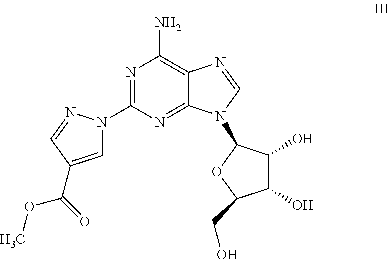 [SMILES: COC(=O)c1cnn(-c2nc(N)c3ncn([C@@H]4O[C@H](CO)[C@@H](O)[C@H]4O)c3n2)c1]
with methylamine in a non-aqueous solvent to produce anhydrous 2-[4-[(methylamino)carbonyl]-1-H-pyrazol-1-yl]adenosine,
b) adding water to the product of step a) to produce of 2-[4-[(methylamino)carbonyl]-1-H-pyrazol-1-yl]adenosine monohydrate of formula I.

2. (canceled)

3. (canceled)

4. (canceled)

5. The method according to claim 1, wherein the non-aqueous solvent is an alcohol or a polar aprotic solvent or a combination thereof.

6. The method according to claim 5, wherein the alcohol is methanol or ethanol.

7. The method according to claim 5, wherein the polar aprotic solvent is dimethyl sulfoxide.
